(19) United States
(12) Patent Application Publication
(10) Pub. No.: US 2025/0029734 A1
(43) Pub. Date: Jan. 23, 2025

(54) SYSTEMS AND METHODS FOR PATIENT DATA MANAGEMENT

(71) Applicant: BioSpark AI Technologies Inc., Vancouver (CA)

(72) Inventors: Kristian THORLUND, Vancouver (CA); Reza JAFAR, Vancouver (CA); Maxime LEROUX, Vancouver (CA)

(21) Appl. No.: 18/776,169

(22) Filed: Jul. 17, 2024

Related U.S. Application Data
(60) Provisional application No. 63/527,723, filed on Jul. 19, 2023.

(51) Int. Cl. G16H 50/70; G16H 10/60; G16H 15/00

(52) U.S. Cl. CPC G16H 50/70; G16H 10/60; G16H 15/00

(57) ABSTRACT

Example embodiments provide systems and methods for managing data. An example method for generating structured metadata from a plurality of published case report comprises: identifying a plurality of relevant case reports; extracting relevant text; generating a plurality of entities, wherein each of the entities has an entity type; generating relationships between the any entity pair; and grouping two or more of the entities into a group based on one or more of: the entity types of one or more of the entities, and one or more of the relationships.

### CROSS-REFERENCE TO RELATED APPLICATIONS

This application claims priority from application No. 63/527,723, filed 19 Jul. 2023. For purposes of the United States, this application claims the benefit under 35 U.S.C. § 119 of application No. 63/527,723, filed 19 Jul. 2023, and entitled SYSTEMS AND METHODS FOR PATIENT DATA MANAGEMENT which is hereby incorporated herein by reference for all purposes.

### TECHNICAL FIELD

The present disclosure is directed to systems and methods for managing patient data. In some embodiments the systems and methods described herein use machine-learning algorithms to generate structured patient data from patient case studies.

### BACKGROUND

Patient case studies are commonly published in medical journals. The case studies comprise a combination of unstructured text and graphics, for example, paragraphs of text interspersed with tables, charts, images and/or the like.

Given the unstructured nature of case studies, the format and content of case studies may vary among medical journals, and even among case studies within the same medical journal. For example, different case studies may include different content, and order similar content differently.

Researchers may analyze case studies to assist in their research. To analyze a case study for a given purpose, a researcher may manually review the case study, identify the content relevant to their purpose, and consider how the relevant content relates to their research. For example, a researcher researching a connection between a set of positive genetic markers and a response to a new cancer therapy may review a given cancer case study to identify if the case study includes information on the genetic markers of interest. The researcher may then consider if and how the cancer patient benefitted from receiving the new cancer therapy.

Researchers may more commonly look to analyze two or more case reports to identify relational patterns across patients. A researcher may, for example, want to investigate whether patients with a specific genetic marker tend to benefit more from a new cancer therapy than patients without the genetic marker. As another example, a researcher researching a connection between a drug and an adverse event, or a researcher researching the possibility of an approved drug providing clinical benefits when used ‘off-label’, i.e. outside of its label.

To analyze two or more case studies, a researcher may manually review multiple case studies, identify the information relevant to their purpose, and then attempt to draw a conclusion from the relevant content of the two or more case studies.

Given the considerable number of available case studies and the unstructured nature of case studies, a researcher may miss a relevant case study, or miss a piece of relevant information in a case study or miss considering how the relevant information relates to their research. Furthermore, manually reviewing case studies is time-consuming and prone to human error and the number of case studies that can be reviewed for a given purpose is limited by the capacity of a human researcher.

There is a general desire for improved methods and systems for identifying relevant information in patient case studies and connections between the relevant information.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools, and methods which are meant to be exemplary and illustrative, not limiting in scope. In various embodiments, one or more of the above-described problems have been reduced or eliminated, while other embodiments are directed to other improvements.

This invention has a number of aspects. These aspects include, without limitation:
systems and methods for managing patient data;
systems and methods for retrieving patient data;
systems and methods for parsing data;
systems and methods for characterizing patient data;
systems and methods for predicting relationships within patient data;
systems and methods for organizing patient data according to a timeline;
systems and methods of mapping medical codes from patient data; and/or
systems and methods for visualizing patient journey data.

Some embodiments of the present invention comprise a method for generating structured metadata from a plurality of published case reports, the method comprising: identifying a plurality of relevant case reports from a database of published case reports; extracting relevant text from one or more of the relevant case reports; extracting a plurality of entities from the relevant text, wherein each of the entities has an entity type and corresponds to at least a part of the relevant text of one of the relevant case reports; predicting the relationship between any pair of extracted entities within a sentence; and grouping two or more of the entities into a group based on one or more of: the entity types of one or more of the entities and their relationship.

In some embodiments, the method further comprises normalizing one or more of the entities. Normalizing one or more of the entities may comprise associating two or more of the entities with a sub-category.

In some embodiments, grouping the two or more of the entities into the group comprises: identifying a head entity for the group; and identifying one or more child entities for the group.

In some embodiments, identifying the head entity for the group comprises identifying one of a plurality of entities with an entity type of greater priority than an entity type of another one of the plurality of entities.

In some embodiments, identifying the child entities for the group comprises identifying one or more of the plurality of entities with an entity type of lower priority than an entity type of another one of the plurality of entities.

In some embodiments, identifying the child entities for the group comprises identifying one or more of the plurality of entities not identified as the head entity.

In some embodiments, the database comprises the OVID™ database and the published case reports comprise medical case reports.

In addition to the exemplary aspects and embodiments described above, further aspects and embodiments will become apparent by reference to the drawings and by study of the following detailed descriptions. It is emphasized that the invention relates to all combinations of the above features, even if these are recited in different claims.

Some embodiment of the present invention provide a method for generating structured metadata from a plurality of published case reports, the method comprising: identifying a plurality of relevant case reports from a database of published case reports; extracting relevant text from one or more of the relevant case reports; extracting a plurality of entities from the relevant text, wherein each of the entities has an entity type and corresponds to at least a part of the relevant text; predicting a relationship between one or more pairs of the extracted entities; grouping two or more of the entities into a group based on the entity types of one or more of the entities and the relationship; and mapping one or more of one or more of the entities and the predicted relationship to a database of medical terminology.

In some embodiments, the database of medical terminology comprises one or more of: the ICD10 database, the SNOMED database, and the NCBI database.

Some embodiments comprise normalizing one or more of the entities.

In some embodiments, normalizing one or more of the entities comprises associating two or more of the entities with a sub-category.

In some embodiments, grouping the two or more of the entities into the group comprises: identifying a head entity for the group; and identifying one or more child entities for the group.

In some embodiments, identifying the head entity for the group comprises identifying one of a plurality of entities with an entity type of greater priority than an entity type of a related plurality of entities.

In some embodiments, identifying the child entities for the group comprises identifying one or more of the plurality of entities with an entity type of lower priority than an entity type of a related plurality of entities.

In some embodiments, identifying the child entities for the group comprises identifying one or more of the plurality of entities not identified as the head entity.

In some embodiments, identifying the plurality of relevant case reports comprises: generating a confidence score for each of the published case reports with a first machine-learning model; and identifying the published case reports with a confidence score above a threshold confidence interval.

In some embodiments, generating the confidence interval comprises: identifying an abstract of each of the published case reports; and generating the confidence score based at least in part from the identified abstract of each of the published case reports.

In some embodiments, the first machine-learning model comprises a trained natural language processing (NLP) model.

In some embodiments, extracting the relevant text comprises: converting one or more of the relevant case reports to a machine-readable file format; and identifying patient data in one or more of the relevant case reports.

In some embodiments, generating the plurality of entities comprises: generating a plurality of tokens from the relevant text; and generating an entity type for each of the tokens using a second machine-learning model.

In some embodiments, predicting the relationship comprises generating the relationship using a third machine-learning model.

In some embodiments, predicting the relationship comprises, for one or more pairs of the entities:
generating a relationship confidence score for each of the pairs of entities with the third machine-learning model; and
predicting the relationship between each of the pairs of entities based at least in part on the relationship confidence score corresponding to the pair of entities.

Some embodiments comprise categorizing the extracted entities into sub-categories using a fourth machine-learning model.

Some embodiments of the present invention provide a method for training a machine-learning model for generating a patient journey from a medical case report, the method comprising: identifying a plurality of relevant case reports from a database of published case reports; extracting relevant text from the relevant case reports; generating a plurality of entities from the relevant text, wherein each of the entities has an entity type and corresponds to at least a part of the relevant text; generating a plurality of relationships between a plurality of pairs of the entities; grouping two or more of the entities into a group based on one or more of: the entity types of one or more of the entities, and one or more of the relationships between two or more of the entities; and training a first machine-learning model with one or more of: the plurality of relevant case reports, the extracted relevant text, the entities, and the relationships.

In some embodiments, the first machine-learning model comprises a BioBERT™ natural language processing model.

In some embodiments, identifying the plurality of relevant case reports comprises: generating a confidence score for each of the published case reports with a second machine-learning model; and identifying the published case reports with a confidence score above a threshold confidence interval.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is a block diagram illustrating a system according to an example embodiment of the invention.

FIG. 2 is a block diagram illustrating a method according to an example embodiment of the invention.

### DESCRIPTION

Throughout the following description, specific details are set forth in order to provide a more thorough understanding to persons skilled in the art. However, well-known elements may not have been shown or described in detail to avoid unnecessarily obscuring the disclosure. Accordingly, the description and drawings are to be regarded in an illustrative, rather than a restrictive, sense.

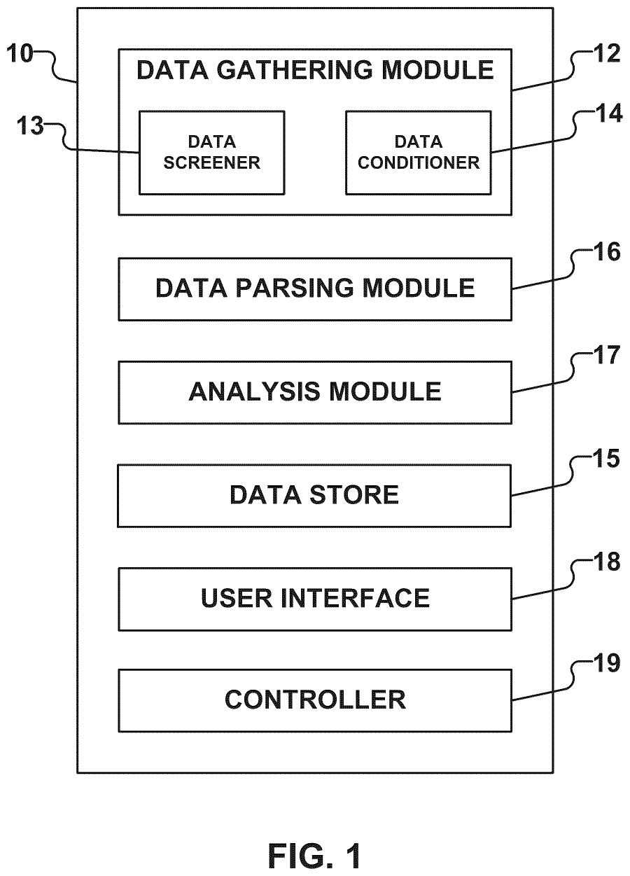

FIG. 1 is a block diagram that schematically illustrates an example system 10 for managing patient data. System 10 gathers and analyzes data such as scientific papers, clinical studies or trials, regulatory approval documents, patient records and/or the like related to one or more specific medical conditions or sub-types of medical conditions. A user (e.g. a doctor, researcher, etc.) may interact with system 10 to access and/or explore desired patient data.

For example, system 10 may manage cancer-related patient data. In some embodiments, system 10 manages patient data related to one or more specific types of cancer (e.g. lung cancer, hematological cancer, breast cancer, colorectal cancer, pancreatic cancer, bladder cancer, etc.). Patient data may be organized into data items (further described below). In some embodiments, the patient data managed by system 10 comprises at least tens of thousands of data items.

System 10 comprises different functional units. The different functional units may be provided as modules. In some embodiments, system 10 is hosted on one or more local computer systems. In some embodiments, system 10 is at least partially hosted on a distributed computing network system.

Patient data items to be managed by system 10 may be searched for and retrieved by data gathering module 12. For example, data gathering module 12 may search through databases (e.g. scientific paper databases, clinical study databases, etc.) for relevant data. In some embodiments data gathering module 12 autonomously searches through databases for relevant data. In some embodiments data gathering module 12 searches through and retrieves data from an online repository of case report data, for example the OVID™ database.

As shown in FIG. 1, data gathering module 12 may comprise data screening sub-module 13 and data conditioning sub-module 14.

Data screening sub-module 13 determines whether a newly located item of data (e.g. a new case report, a new scientific paper, a new clinical study, etc.) should or should not be gathered for system 10. Data screening sub-module 13 may base its decision on whether the newly located item of data should or should not be gathered for system 10 based on the prediction of a trained machine learning model.

In some embodiments, data screening sub-module 13 comprises a trained machine learning model which autonomously selects which data items to include and which data items to exclude. In some embodiments, data screening sub-module 13 determines whether to include or exclude a data item based on an analysis of only a portion of the data item. Such analysis may be quick. For example, if the data item being searched for includes a scientific paper, the machine learning model of data screening sub-module 13 may be trained to predict a new scientific paper's relevance (i.e. whether it should or should not be included) based on its title and abstract (or a portion thereof). In some embodiments, the machine learning model comprises a natural language processing (NLP)-based model that is trained to determine the inclusion or exclusion of the data based on the text of its title and abstract.

Data conditioning sub-module 14 conditions new data items located by data gathering module 12 for further processing and management by system 10 (if conditioning of the new data items is required). Conditioning the new data items may include, for example, any one or more of the following:
converting data from one format to another (e.g. converting a PDF document to JSON (JavaScript Object Notation), etc.);
extracting relevant portions of the data so that only the relevant portions of the data are further processed (e.g. extracting only the case report section from an XML case report publication, extracting only the case report section from an HTML case report publication, etc.); and/or;
the like.

The patient data may be stored in at least one data store 15. New data items may be stored in data store 15 as they are located and retrieved. If a data item is later determined to be irrelevant, such data item may be flagged in data store 15 so that it would be excluded upon data searches.

In some embodiments of data store 15:
data store 15 comprises an inverted index mapping, wherein the index maps key search terms and/or entity types to case report identification numbers;
data store 15 stores data in an unstructured JSON format;
data store 15 stores multiple versions of one or more data items; and/or
data store 15 comprises a dynamic lookup system mapping entities to all available values.

One or more of the above features of data store 15 may provide improved speed and/or efficiency for one or both of searching data store 15 and retrieving data from data store 15 over embodiments of data store 15 without the one or more features.

Additionally, or alternatively, patient data to be managed by system 10 may be pre-loaded onto data store 15.

Data parsing module 16 autonomously parses a data item into one or more sections. For example, if the data item is a scientific paper, data parsing module 16 may parse each sentence of the scientific paper into a section. A section may comprise a plurality of tokens (words or sub-words). Each token may, for example, be labelled using natural language processing or the like. Tokens or groups of tokens may be categorized into categories. Post-processing may generate structured data comprising key data entities (e.g. categories) and more specific information related to each of the data entities (e.g. sub-categories). The post-processing may, for example, be based on the performed labelling. In some embodiments data parsing module 16 parses the data item sequentially (e.g. sentence-by-sentence). In some embodiments data parsing module 16 parses the data items non-sequentially (e.g. locates the most relevant sentences first and parses those sentences first).

In some embodiments, data parsing module 16 autonomously characterizes the parsed sections of the data item into a category. In some embodiments, a plurality of portions of a section are characterized into one category. The categories may, for example, comprise characteristics, primary categories, secondary categories and dependent categories. Characteristics may relate to patient characteristics and may, for example, comprise characteristics such as: age, sex, ethnicity, smoking, other risk factors, comorbidities, and/or the like.

The types of different relations which may be characterized comprise one or more of: ‘related’ (general association), abbreviation, time anchor, sub type, extension, reference, history, cause and effect, and/or the like.

As illustrative non-limiting examples:
a general entity category may be further characterized into which of the other primary categories it represents the best;
a diagnosis category may be further characterized into which type of cancer it is most likely to represent;
an outcome category may be further characterized into one of a set number (e.g. 5) major outcome categories it is most likely to represent (e.g. survival or tumor response, etc.); and/or
the like.

Primary categories may be independent main categories. Primary categories may, for example, comprise one or more of: clinical visit, diagnosis, metastasis, outcome, adverse event, biomarker, genetic mutation, drug treatment, radio therapy, surgery, concomitant treatment, other treatment, vitals and/or the like. Secondary categories may be independent or dependent on one or more primary categories.

Secondary categories may, for example, comprise one or more of: drug class, treatment line, stage (of cancer diagnosis), criteria (e.g. for clinical outcome or adverse event definition), and/or the like. Dependent categories are related to a primary or secondary category. Dependent categories may, for example, comprise one or more of: negation modifier, unknown modifier, change modifier, quantity, positivity indicator, negativity indicator, anatomical location, details, status, specimen, treatment event, and/or the like. Additionally, three categories comprising ‘time point’, ‘duration’ and ‘history’ are labels that may be used to represent time. Any primary or secondary category may be related to each other as well as a dependent category or time category. Dependent categories are related to primary or secondary categories.

The characterization of the data sections (or portions of a data section) into categories may, for example, be based on a likelihood of a data section (or portion of a data section) corresponding to a particular category. In some embodiments, a data section (or portion of a data section) is characterized into the category which has the highest likelihood of corresponding to the data section (or portion of the data section).

Data parsing module 16 may comprise a trained machine learning model. The machine learning model may be trained to autonomously parse and characterize parsed sections (or portions of sections) of the data items of the patient data. In some embodiments the trained machine learning model comprises a natural language processing based model.

In some embodiments, data parsing module 16 parses and/or characterizes the data items, sections, or portions of sections thereof using a machine-learning model. The machine-learning model can be updated after re-training on larger samples of data. Where data parsing module 16 parses and/or characterizes the data items using a newer model version, data store 15 may store multiple versions of the data items corresponding to different model versions.

In some embodiments, data parsing module 16 may comprise deterministic enhancement of the data items, sections, or portions of sections thereof. Deterministic enhancement, for example, may comprise mapping sections of the data to a database of medical terminology, for example, one or more of: the International Classification of Diseases (ICD) database, the structured clinical vocabulary for use in an electronic health record (SNOMED) database, and the like. In some embodiments, the deterministic enhancement may comprise using a trained machine learning algorithm.

In some embodiments data parsing module 16 may exclude from characterization sections (or portions of sections) which are deemed to be irrelevant. In some embodiments the one or more categories comprise a category for irrelevant data. Data parsing module 16 may characterize sections which are deemed to be irrelevant into the irrelevant data category. In some embodiments dependent categories such as “negation modifier” and “unknown modifier” may be used to characterize irrelevant data. In some embodiments an additional machine learning model may be trained to characterize the negation modifier and/or the unknown modifier. In some embodiments the one or more categories comprise a generic data category intended to capture data sections which are not characterized into any of the other categories.

In one example case relating to cancer data, the one or more categories may comprise the following categories:
age
smoking status;
sex;
test type (e.g. imaging, lab study, clinical examination, physical/bodily function, etc.);
clinical outcome;
clinical endpoint;
cancer diagnosis stage;
cancer type;
cancer location (e.g. an anatomical location);
treatments (e.g. drug therapy, radio therapy, surgery, other);
modifiers (e.g. negation, unknown, change modifiers);
disease history; and/or
the like.

In some embodiments data parsing module 16 further autonomously characterizes sections (or portions of sections) of the data item into one or more sub-categories of a category. The sub-categories may relate to more specific characteristics of the general category. For example, if a section of data is characterized as relating to sex, data parsing module 16 may further characterize that section of data as relating to female or male. As another example, if a section of data is characterized as relating to smoking status, data parsing module 16 may further characterize that section of data as relating to a current smoker, past smoker or never smoker.

In some embodiments each category may comprise a plurality of sub-categories. For example, a cancer location category may comprise a plurality of sub-categories such as lung, breast, colon, pancreas, liver, brain, etc. In some embodiments at least one category comprises a plurality of sub-categories and at least one category does not comprise any sub-categories. In some cases none of the categories comprise a sub-category.

In some embodiments one or more of the machine learning models of data parsing module 16 described above are further trained to characterize sections of data into sub-categories. In some embodiments data parsing module 16 comprises one or more additional trained machine learning models which are trained to characterize sections of data into sub-categories. The one or more additional machine learning models may each comprise a natural language processing based model.

In some embodiments one or more of the machine learning models of data parsing module 16 are trained with a method of supervised learning. For example, training data may comprise several thousand labeled data entries.

In some embodiments, data parsing module 16 comprises a supervised Named Entity Recognition (NER) algorithm, wherein the NER algorithm extracts entities of interests by categorizing tokens into different labels (e.g., age, diagnosis, outcome, and the like). The input to the NER algorithm may comprise a sentence, and the output of the NER algorithm may comprise a list of extracted entities with their labels.

In some embodiments, data parsing module 16 comprises a multi-classification (MC) algorithm. The inputs to the MC algorithm may comprise an entity text (one or multiple tokens), and the output may comprise one of a plurality of pre-defined sub-categories.

One or both of the NER algorithm and the MC algorithm may comprise a BioBERT model. Where both the NER algorithm and the MC algorithm comprise a BioBERT model, one or more weights of the two BioBERT models may be in common between the NER algorithm and the MC algorithm. In some embodiments, the BioBERT model of the NER algorithm and the BioBERT model of the MC algorithm may comprise the same weights in all neural network layers but the last neural network layer.

In some embodiments the searching uses standardized international medical terms. For example, the searching may match ICD-10 terms and codes. As another example, the searching may match SNOMED codes.

Analysis module 17 may autonomously predict relationships between sections of data, and/or generate groups from the data.

Some non-limiting examples of relationship types that may be identified by analysis module 17 include:
negative, meaning no relationship between a pair of entities;
abbreviation, meaning that one entity is an abbreviated form of another entity;
reference, meaning one entity refers to another entity, generally a previous entity;
sub type, meaning one entity is a sub type of another entity; for example, when a drug treatment is a sub type of a drug class; and
cause and effect, meaning one entity happened due to the presence of another entity.

In some embodiments analysis module 17 comprises at least one machine learning model. The machine learning model may be trained to recognize or predict a relationship between sections of data.

Once analysis module 17 generates one or more relationships, analysis module 17 may group two or more similar entities into a group. Each group includes a time point, a head entity and a list of children entities. The head entity identifies the group and the children provide extra information about the head. For example, if a diagnosis entity is related to a stage entity, a group is created from the two entities, wherein the head comprises the diagnosis entity, and the one or more child entities comprise the stage entity.

In some embodiments of analysis module 17, the entity groups are generated with a trained machine-learning algorithm. The trained machine-learning algorithm may be trained on training data comprising:
for each entity pair in any annotated sentence: generating a modified version of the sentence by adding one or more special characters before and/or after each entity to create inputs; and
generating outputs from the annotated relationship for the pair.

Modules 16 and 17 may extract entities and predict relationships within only one sentence. In order to group individual entities into coherent groups, capture information across sentences, and remove redundant information, a set of post-processing rules may be applied to the data. The set of rules may comprise: identifying a list of entity labels that may be the head of a group ranked by entity importance. For example, negative modifier, unknown modifier, stage, or treatment event, cannot be a head entity. Some of the rules may include:
if in a sentence a stage entity is extracted with no relationship, relate it to the last diagnosis entity found in previous sentences (if any);
if no time point is mentioned in a sentence, it inherits the latest timepoint from previous sentences;
if an extracted drug treatment is not related to a treatment event entity, remove it (do not make a group from it); and/or
the like.

A user (or users) may interact with system 10 through user interface (“U/l”) module 18. For example, U/I module 18 may comprise one or more user inputs. The user inputs may provide an interface for a user to select patient data which they would like to retrieve. For example, a user may input keywords related to the patient data they would like to retrieve, select the data they would like to retrieve through a graphical user interface (“GUI”), etc.

U/I module 18 also comprises one or more outputs for outputting patient data, predicted outcomes, data relationships, etc. For example, U/I module 18 may comprise at least one display. In some cases, patient outcomes or patient journeys (e.g. patient treatment journeys) may be visually displayed. In some cases, system 10 generates graphical illustrations such as Gantt charts for illustrating a patient's journey.

In some embodiments, a user interacts with system 10 via a web interface. Provided the user creates an account, the user can interact with the system via a search interface allowing them to efficiently search data store 15 with criteria such as: cancer location, diagnosis, biomarkers, mutations, treatments, adverse events, outcomes, and the like; all of which using instantaneous, auto-complete-enabled search fields spanning the entire enhanced dataset. Once a user has identified a subset of interest, the user can visualize the data summarizing each case report in the form of a summary case report data (Sex, Age, Genomics, Biomarkers & others) and a patient-journey Gantt chart visual representation. The Gantt charts are filterable on entity types and provide a timeline of the patient's journey with enhanced data structured in an easily digestible format.

As shown in FIG. 1 system 10 also comprises at least one controller 19. Controller 19 controls the different functional units or modules of system 10. In some embodiments, one or more functional units or modules of system 10 each comprise a controller. The controller of a functional unit or module may interface with controller 19 and/or other controllers of system 10.

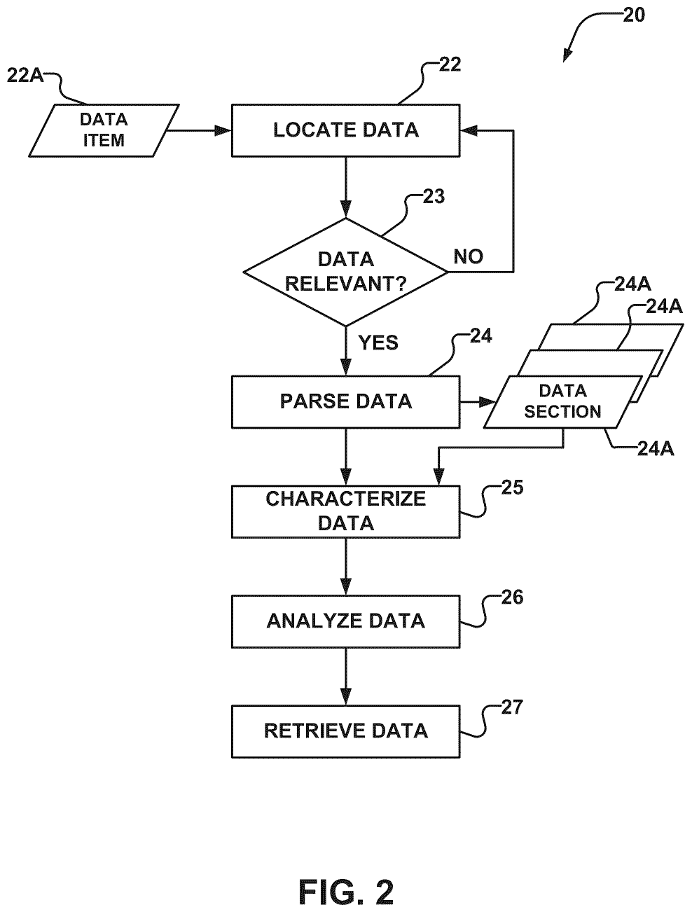

FIG. 2 is a block diagram which illustrates an example method 20 for managing patient data.

In block 22, a new data item 22A is located (e.g. a scientific paper, clinical study, medical record, etc.).

Block 23 determines whether new data item 22A is relevant. As described elsewhere herein, relevance of new data item 22A may be determined based on only a portion of data item 22A (e.g. the title and abstract of data item 22A, and/or the like). If new data item 22A is not relevant, then method 20 returns to block 22, and performs block 22 for a further new data item 22A′. Otherwise method 20 proceeds to block 24.

In block 24 new data item 22A is parsed into data sections 24A. For example, each sentence of new data item 22A may be parsed into a section 24A.

In block 25 parsed sections 24A are characterized. In some embodiments parsed sections 24A are characterized into one or more categories. In some embodiments a parsed section 24A is further characterized into one or more sub-categories of a category.

In block 26 relationships between different sections of data may be determined as described elsewhere herein. Additionally, entity groups are created using related sections of data and pre-defined post-processing rules.

A user may retrieve desired patient data (e.g. from system 10) in block 27. Patient data which is retrieved may at least partially be based on entities extracted in block 25 and relationships predicted in block 26.

Throughout the description herein various functional units or modules of system 10 determine relevance of data. Relevance of data may, for example, be determined based on a comparison of a likelihood of data corresponding to a desired thing compared to a threshold likelihood. For example, data may be relevant if it has a likelihood of corresponding to a widget that is greater than a set threshold likelihood.

A natural language processing model described herein may comprise models created using BioBERT™. BioBERT (Bidirectional Encoder Representations from Transformers for Biomedical Text Mining) is a pre-trained biomedical language model which has shown a great performance on biomedical NLP tasks such as named entity recognition, relation extraction and question answering. For each desired task, the model needs to be fine-tuned using an annotated training set. The higher the quality and quantity of a training set, usually the higher the accuracy of the model.

### Some Embodiments

One or more embodiments of the present invention may comprise one or more of:
a computer system configured to perform one or more of the methods disclosed herein; and
a computer readable memory storing machine-readable instructions that when performed by a computer system cause the computer system to perform one or more of the methods disclosed herein.

One or more embodiments of the present invention are described as comprising one or more models. As used herein, a model may comprise any combination of computer hardware and computer software configured to provide the described functionality. For example, a model may comprise:
a sequence of computer instructions;
a look-up table; and
a trained machine-learning algorithm.

### Interpretation of Terms

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms.

Embodiments of the invention may be implemented using specifically designed hardware, configurable hardware, programmable data processors configured by the provision of software (which may optionally comprise “firmware”) capable of executing on the data processors, special purpose computers or data processors that are specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”). Examples of programmable data processors are: microprocessors, digital signal processors (“DSPs”), embedded processors, graphics processors, math co-processors, general purpose computers, server computers, cloud computers, mainframe computers, computer workstations, and the like. For example, one or more data processors in a control circuit for a device may implement methods as described herein by executing software instructions in a program memory accessible to the processors.

Processing may be centralized or distributed. Where processing is distributed, information including software and/or data may be kept centrally or distributed. Such information may be exchanged between different functional units by way of a communications network, such as a Local Area Network (LAN), Wide Area Network (WAN), or the Internet, wired or wireless data links, electromagnetic signals, or other data communication channel.

For example, while processes or blocks are presented in a given order, alternative examples may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, or may be performed at different times.

In addition, while elements are at times shown as being performed sequentially, they may instead be performed simultaneously or in different sequences. It is therefore intended that the following claims are interpreted to include all such variations as are within their intended scope.

The invention may also be provided in the form of a program product. The program product may comprise any non-transitory medium which carries a set of computer-readable instructions which, when executed by a data processor, cause the data processor to execute a method of the invention. Program products according to the invention may be in any of a wide variety of forms. The program product may comprise, for example, non-transitory media such as magnetic data storage media including hard disk drives, optical data storage media including CD ROMs, DVDs, electronic data storage media including ROMs, flash RAM, EPROMs, hardwired or preprogrammed chips (e.g., EEPROM semiconductor chips), nanotechnology memory, or the like. The computer-readable signals on the program product may optionally be compressed or encrypted.

In some embodiments, the invention may be implemented in software. For greater clarity, “software” includes any instructions executed on a processor, and may include (but is not limited to) firmware, resident software, microcode, and the like. Both processing hardware and software may be centralized or distributed (or a combination thereof), in whole or in part, as known to those skilled in the art. For example, software and other modules may be accessible via local memory, via a network, via a browser or other application in a distributed computing context, or via other means suitable for the purposes described above.

Where a component (e.g. a model, a software module, processor, assembly, device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

Various features are described herein as being present in “some embodiments”. Such features are not mandatory and may not be present in all embodiments. Embodiments of the invention may include zero, any one or any combination of two or more of such features. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible).

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

1. A method for generating structured metadata from a plurality of published case reports, the method comprising:
identifying a plurality of relevant case reports from a database of published case reports;
extracting relevant text from one or more of the relevant case reports;
extracting a plurality of entities from the relevant text, wherein each of the entities has an entity type and corresponds to at least a part of the relevant text;
predicting a relationship between one or more pairs of the extracted entities;
grouping two or more of the entities into a group based on the entity types of one or more of the entities and the relationship; and
mapping one or more of one or more of the entities and the predicted relationship to a database of medical terminology.

2. The method according to claim 1, wherein the database of medical terminology comprises one or more of: the ICD10 database, the SNOMED database, and the NCBI database.

3. The method according to claim 1, further comprising normalizing one or more of the entities.

4. The method according to claim 3, wherein normalizing one or more of the entities comprises associating two or more of the entities with a sub-category.

5. The method according to claim 1, wherein grouping the two or more of the entities into the group comprises:
identifying a head entity for the group; and
identifying one or more child entities for the group.

6. The method according to claim 5, wherein identifying the head entity for the group comprises identifying one of a plurality of entities with an entity type of greater priority than an entity type of a related plurality of entities.

7. The method according to claim 5, wherein identifying the child entities for the group comprises identifying one or more of the plurality of entities with an entity type of lower priority than an entity type of a related plurality of entities.

8. The method according to claim 5, wherein identifying the child entities for the group comprises identifying one or more of the plurality of entities not identified as the head entity.

9. The method according to claim 1, wherein the database comprises the OVID™ database and the published case reports comprise medical case reports.

10. The method according to claim 1, wherein identifying the plurality of relevant case reports comprises:
generating a confidence score for each of the published case reports with a first machine-learning model; and
identifying the published case reports with a confidence score above a threshold confidence interval.

11. The method according to claim 10, wherein generating the confidence interval comprises:
identifying an abstract of each of the published case reports; and
generating the confidence score based at least in part from the identified abstract of each of the published case reports.

12. The method according to claim 10, wherein the first machine-learning model comprises a trained natural language processing (NLP) model.

13. The method according to claim 1, wherein extracting the relevant text comprises:
converting one or more of the relevant case reports to a machine-readable file format; and
identifying patient data in one or more of the relevant case reports.

14. The method according to claim 1, wherein generating the plurality of entities comprises:
generating a plurality of tokens from the relevant text; and
generating an entity type for each of the tokens using a second machine-learning model.

15. The method according to claim 1, wherein predicting the relationship comprises generating the relationship using a third machine-learning model.

16. The method according to claim 15, wherein predicting the relationship comprises, for one or more pairs of the entities:
generating a relationship confidence score for each of the pairs of entities with the third machine-learning model; and
predicting the relationship between each of the pairs of entities based at least in part on the relationship confidence score corresponding to the pair of entities.

17. The method according to claim 1, further comprising categorizing the extracted entities into sub-categories using a fourth machine-learning model.

18. A method for training a machine-learning model for generating a patient journey from a medical case report, the method comprising:
identifying a plurality of relevant case reports from a database of published case reports;
extracting relevant text from the relevant case reports;
generating a plurality of entities from the relevant text, wherein each of the entities has an entity type and corresponds to at least a part of the relevant text;
generating a plurality of relationships between a plurality of pairs of the entities;
grouping two or more of the entities into a group based on one or more of: the entity types of one or more of the entities, and one or more of the relationships between two or more of the entities; and
training a first machine-learning model with one or more of: the plurality of relevant case reports, the extracted relevant text, the entities, and the relationships.

19. The method according to claim 18, wherein the first machine-learning model comprises a BioBERT™ natural language processing model.

20. The method according to claim 18, wherein identifying the plurality of relevant case reports comprises:
generating a confidence score for each of the published case reports with a second machine-learning model; and
identifying the published case reports with a confidence score above a threshold confidence interval.
